(19) United States
(12) Patent Application Publication
(10) Pub. No.: US 2026/0301895 A1
(43) Pub. Date: Oct. 1, 2026

(54) METHOD AND APPARATUS FOR UNLINKING A REPORT FROM AN EXAMINATION

(71) Applicants: TOPCON CORPORATION, Tokyo (JP); Topcon Healthcare, Inc., Oakland, NJ (US)

(72) Inventors: Carolin Tanja SEITH, Linkenheim-Hochstetten (DE); Hanna Kaarina KINNUNEN, Helsinki (FI)

(73) Assignees: TOPCON CORPORATION, Tokyo (JP); Topcon Healthcare, Inc., Oakland, NJ (US)

(21) Appl. No.: 19/095,205

(22) Filed: Mar. 31, 2025

(51) Int. Cl. G16H 10/60

(52) U.S. Cl. CPC G16H 10/60

(57) ABSTRACT

A method for managing AI analysis of medical test results (e.g., examination parameters) comprises the step of receiving user input requesting AI analysis of examination parameters. AI analysis of the examination parameters is initiated in response to the receiving user input requesting AI analysis of examination parameters to generate an AI report. The AI report is then stored in memory and is linked with an examination., unlinking The AI report is unlinked from the examination in response to receiving a delete request before the examination has been saved while maintaining the AI report in memory.

### FIELD OF THE INVENTION

The present disclosure relates generally to a method and apparatus for managing artificial intelligence (AI) reports, and more particularly, for managing AI reports that were generated by AI functions using examination parameters.

### BACKGROUND

Eye care providers have varying levels of experience. Not all eye care providers are capable of analyzing data output by examination equipment. Artificial intelligence (AI) can used to analyze medical test results (e.g., examination parameters) and provide insights and/or recommendations to eye care providers. Such insights and/or recommendations can include medical records, such as clinical findings or examination results, and the obligation or necessity of their storage that is governed by the laws and regulations of each country. In addition, although the implementation of artificial intelligence analysis for medical test results could potentially involve in-house development and integration, it is more likely that externally developed solutions will be integrated or externally operated services will be utilized, considering current and future trends. This is because numerous entities exist that support AI-based analysis of data. However, utilizing such external AI-based analysis may incur costs. Further, charges may be incurred even if the analysis is stopped before being completed. In addition, charges may be incurred for analyzing the same medical test results using the same AI functions. What is needed is a method and apparatus to manage the AI analysis of examination parameters especially the generation and storage of AI reports. In addition, method and apparatus should also limit and reduce the costs of using AI analysis.

### SUMMARY

A method for managing AI analysis of medical test results (e.g., examination parameters) comprises the step of receiving user input requesting AI analysis of examination parameters. AI analysis of the examination parameters is initiated in response to the receiving user input requesting AI analysis of examination parameters to generate an AI report. The AI report is then stored in a storage and is linked with an examination record wherein a link is created to manipulate the AI report. The AI report is unlinked from the examination record by deleting the link while maintaining the AI report in storage in response to receiving a delete request of the AI report before the examination record has been saved. In one embodiment, the AI analysis is terminated in response to receiving a terminate request before an AI report has been received. In one embodiment, terminating the AI analysis is prohibited in response to receiving a terminate request after an AI report has been received. In one embodiment, unlinking of the AI report from the examination record after the examination has been saved is prohibited. In one embodiment, the prohibiting comprises deactivating a delete AI report icon. In one embodiment, the link is represented with a thumbnail image of AI report on which a delete AI report icon is displayed while the AI report icon is being hovered over with by a pointing device. In one embodiment, a same set of examination parameters that were used to generate a previous AI report using a particular AI function are prohibited from being used to generate the previous AI report again using the particular AI function. In one embodiment, the same set of examination parameters are prohibited from being used by disabling selection of the same set of examination parameters that have been previously used to generate a previous AI report. In one embodiment, examination parameter icons representing the same set of examination parameters that have been previously used to generate the previous AI report each have an identifier indicating that that examination parameter was previously used with the particular AI function to generate the previous AI report. In one embodiment, the same set of examination parameters are prohibited from being used by disabling selection of the particular AI function used to generate a previous AI report when the same set of examination parameters previously selected to generate the AI report are selected. In one embodiment, an AI function to generate the AI report is automatically selected based on the examination parameters to be analyzed. In one embodiment, the method further includes storing the AI report in a storage located at a different premises, monitoring the deletion of AI reports at both premises, and restoring the AI report when detecting the deletion of the AI reports in either of the premises by copying the AI report remained in the storage to another. In one embodiment, the method further includes storing the AI report in a write-only storage. In one embodiment, the write-only storage is implemented by using a block chain technology wherein the AI report is appended as a block to existing blocks with the hash value of the previous block.

An apparatus having memory storing computer program instructions for managing AI analysis of medical test results and a computer readable medium storing instructions for managing AI analysis of medical test results are also described herein.

### BRIEF DESCRIPTION OF THE DRAWINGS

FIG. 1 shows a clinical decision support system (CDSS) in communication with various examination devices according to one embodiment;

FIG. 2 shows the CDSS of FIG. 1 along with its configuration with respect to other hardware and software components;

FIG. 3 shows a high-level schematic of a computer for implementing method and systems described herein;

FIG. 4 shows a method for opticians to use AI in order to assist in performing a patient's eye health exam;

FIG. 5 shows data flow for image analysis indicating how data flows among a user, data management software, and an AI product according to one embodiment;

FIG. 6 shows a flowchart of a method according to one embodiment;

FIGS. 7A-7C show a screen flow of a user selecting examination parameters for analysis by an AI function according to one embodiment;

FIGS. 8A-8F shows a screen flow of a user selecting an AI function to analyze examination data with the user requesting to stop the analysis before it is finished according to one embodiment;

FIGS. 9A-9C show a screen flow of generation and display of a report according to one embodiment;

FIG. 10 shows a report that is generated by an AI function and displayed to a user according to one embodiment;

FIGS. 11A-11C shows how a user is restricted from generating a report using the same examination parameters and AI function as those used to create a previous report according to one embodiment;

FIGS. 12A-12D shows screen flows pertaining to how a user can request to delete a report before analysis is completed according to one embodiment;

FIGS. 13A-13D show screen flows pertaining to how a user can delete an AI report before an examination is finished according to one embodiment;

FIG. 14 shows data flow among a user, a service operator, and a AI provider according to one embodiment;

FIG. 15 shows data flow among a user, a service operator, and an AI provider when a user chooses to run an AI analysis but then cancels that analysis according to one embodiment;

FIG. 16 shows data flow between a service operator and an AI provider in which data is restored when deleted after a report is made according to one embodiment;

FIG. 17 shows a hardware configuration according to one embodiment;

FIG. 18 shows a hardware configuration according to one embodiment;

FIG. 19 shows a hardware configuration according to one embodiment;

FIG. 20 shows a hardware configuration according to one embodiment;

FIG. 21 shows a hardware configuration according to one embodiment;

FIG. 22 shows a hardware configuration according to one embodiment;

FIG. 23 shows a hardware configuration according to one embodiment; and

FIG. 24 shows an application selection function according to one embodiment.

### DETAILED DESCRIPTION

One way to improve the usability of data pertaining to medical test results is to analyze that data using artificial intelligence (AI) techniques (also referred to as AI or AI functions) to provide insights or recommendations. The term AI, as used herein, pertains to any technique that enables computers to mimic human intelligence using logic, if-then rules, decision trees, and/or machine learning (including deep learning). Machine learning as used herein pertains to a subset of AI that includes statistical techniques that enable machines to improve at tasks with experience. Deep learning as used herein pertains to a subset of machine learning comprising algorithms that permit software to perform tasks, such as speech and image recognition, by using multi-layered neural networks to analyze vast amounts of data.

AI techniques can process large amounts of data and identify patterns, trends, anomalies, or correlations that may not be obvious to human experts. However, AI techniques may also have limitations or uncertainties, such as data quality, algorithm reliability, or ethical issues. Therefore, there is also a need for a method that can transparently and effectively communicate the results of AI analysis to users and allow them to verify, validate, or challenge the result or process of the AI analysis. The methods described herein allow a user to compare the data of medical test results with AI analysis data and to examine the rationale, validity, and/or accuracy of the AI analysis. The methods described herein also improve the usability, transparency, and trustworthiness of medical test results and AI analysis. The methods described herein allow users to conceal AI reports from user interfaces from a usability perspective by providing an unlink function, instead of physically deleting AI reports, while preserving AI reports to ensure the protection of medical and billing records. These methods can control unlinking in relation to the examination record. These methods prevent the deletion and tampering of the AI reports necessary for aligning billing information with the external systems operated by AI providers. These methods help prevent users from incurring additional costs and avoid duplicate charges by controlling duplicate analysis using previously used examination parameters from the perspective of streamlining of the billing.

In one embodiment, data management software comprises a plurality of modules for acquiring, storing, analyzing, and displaying various patient data. Each of the plurality of modules can be enabled or disabled for a particular entity or user of the data management software. The present disclosure describes a timeline function of data management software including a glaucoma clinical decision support system (CDSS), but the disclosure also supports timeline functions for other clinical decision support systems for other types of medical conditions, medical issues, etc. In one embodiment, the CDSS is a module of (i.e., a part of) the data management software and is used to estimate a patient's glaucoma risk score. The risk score is based on well-known algorithms. The purpose of the score, in one embodiment, is not to diagnose glaucoma but instead to help a user, such as a primary health care provider, to determine if there is a need for further examination or evaluation. In one embodiment, the algorithms that can be used for risk calculations include multi-factoral optical coherence tomography (OCT) screening score (MOS), glaucoma health score (GHS), and ocular hypertension treatment study (OHTS).

In one embodiment, each algorithm uses the patient's examination data (i.e., data of medical test results) to calculate a risk score that will indicate the likelihood the patient will develop a glaucoma. The risk score can help a user make an informed decision regarding the patient's care and the next steps.

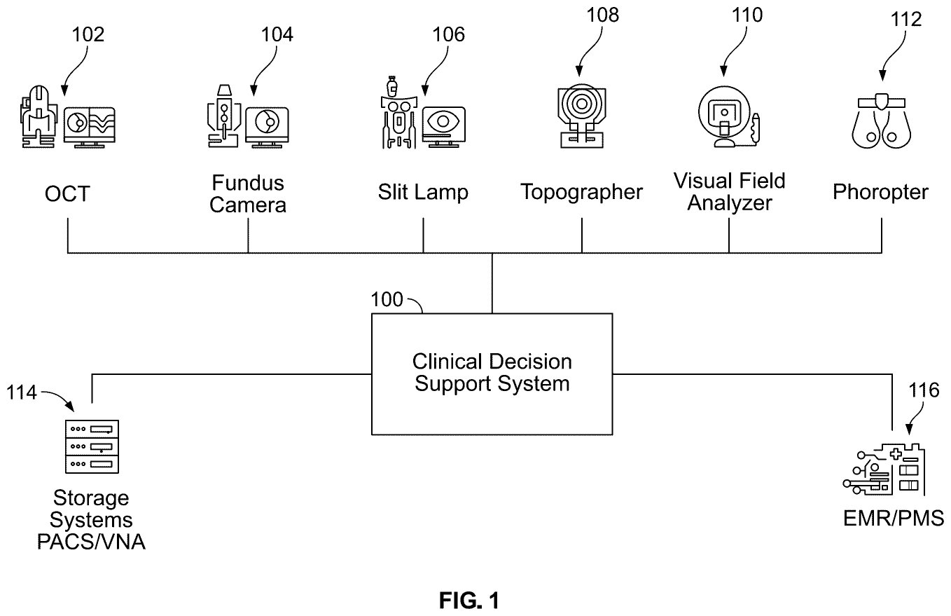

FIG. 1 shows a clinical decision support system (CDSS) 100 in communication with various examination devices including optical coherence tomography (OCT) device 102, fundus camera 104, slit lamp 106, topographer 108, visual field analyzer 110, and phoropter 112, all of which may be used to generate examination parameters during an examination of a patient's eye. CDSS 100 is also in communication with storage systems 114 comprising a picture archiving and communication system (PACS) and a vendor neutral archive (VNA). CDSS 100 is also in communication with electronic medical records (EMR) and practice management software (PMS) 116 which allows a user to access and update EMRs. Data received from examination devices 102-112 along with patient data from storage systems 114 and EMR/PMS 116 allows a user accessing clinical decision support system to display, review, and manage patient health data. The CDSS also assists a user in the selection and application of algorithms with respect to patient health data to determine a patient's condition and if that condition requires a referral to another doctor, such as a specialist. CDSS 100 can also be used for gathering and analyzing data in order to promote efficient and accurate decisions regarding a patient's health.

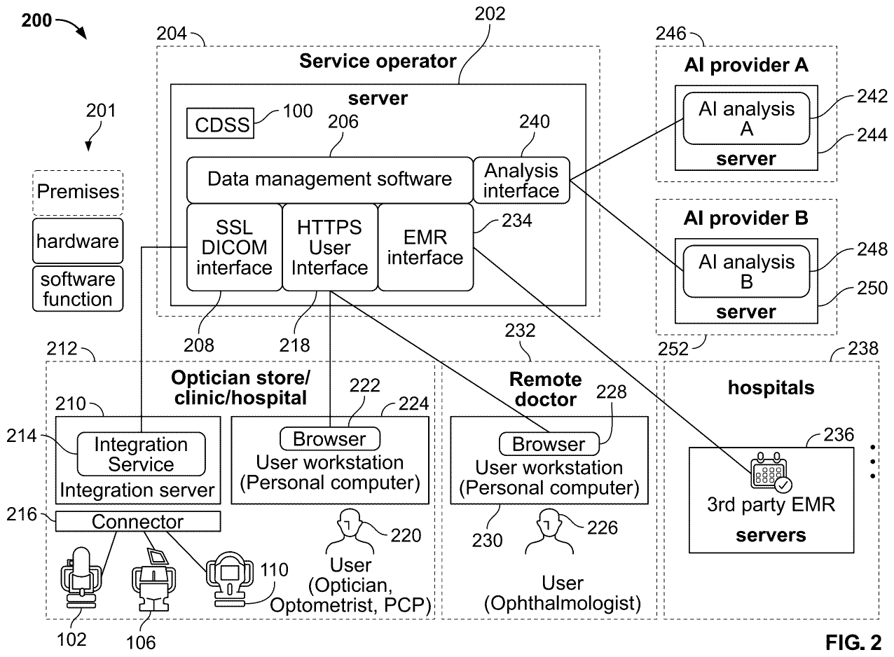

FIG. 2 shows CDSS 100 and its overall logical configuration and functionality with respect to other hardware and software in patient health system 200. It should be noted that premises are identified in FIG. 2 by dashed line border, hardware is identified by a bold solid line border, and a software function is identified by a thin line border as shown by legend 201. CDSS 100 is implemented on server 202 which is implemented in the premises of Service operator 204, who manages and delivers a clinical decision support service for users collaborating with AI providers A, B, and so on. CDSS 100 is a module of data management software 206 which is also implemented on server 202. Data management software 206 is in communication with SSL DICOM interface 208, HTTPS user interface 218, EMR interface 234, and analysis interface 240 all of which are implemented on server 202. In one embodiment, CDSS 100, SSL DICOM interface 208, HTTPS user interface 218, EMR interface 234, and analysis interface 240 are implemented in data management software 206, however one or more of CDSS 100, SSL DICOM interface 208, HTTPS user interface 218, EMR interface 234, and analysis interface 240 can be implemented on server 202 as standalone modules. SSL DICOM interface 208 is in communication with integration server 210 located at optician store 212 (which alternatively may be a clinic or hospital).

Integration server 210 operates integration service 214 which receives data from devices including OCT device 102, slit lamp 106, and visual field analyzer 110 via connector 216. It should be noted that integration service can receive data from additional devices, such as those shown in FIG. 1, although only three devices are shown in FIG. 2 for clarity.

HTTPS user interface 218 allows user 220 located at optical store 212 to access data using browser 222 operated on user workstation 224 (also referred to as personal computer) which are also located at optical store 212. User 220 refers to individuals such as opticians or optometrists working in optical stores, who are proficient in ophthalmic measurements but are not authorized to diagnose ophthalmic diseases. Alternatively, it may refer to primary care physicians (PCPs) who provide initial consultations and health check-ups but lack expertise in diagnosing specialized conditions. HTTPS user interface 218 also allows user 226 located at remote doctor location 232 to access data using browser 228 operated on user workstation 230 (e.g., browser 228 and workstation 230 together form a client and the client can include/support other types of software) which are also located at remote doctor location 232. User 226 refers to specialists such as ophthalmologists who are qualified to perform precise examinations related to ophthalmology or other medical specialists such as cardiologists and neurologists capable of diagnosing specific diseases. In one embodiment, browsers 224 and 228 may be dedicated terminal software or general-purpose client software designed to communicate with data management software 206.

Electronic medical record (EMR) interface 234 is implemented on server 202 and allows data management software 206 to communicate with 3^{rd} party EMR implemented on 3^{rd} party EMR servers 236 operated at hospitals 238. In one embodiment, EMRs are used for data exchange. For example, a remote EMR, such as 3^{rd} party EMR communicates with data management software 206. EMRs can also be sent to other doctors in order to obtain additional opinions.

In one embodiment, CDSS 100 has analysis software (e.g., artificial intelligence analysis software) and can analyze images and other patient data using artificial intelligence or other types of analysis software. CDSS 100 can also transmit information to other locations for analysis using artificial intelligence or other types of analysis software. Analysis interface 240 is implemented on server 202 and allows data management software 206 to communicate with AI provider A 246 and AI provider B 252, who respectively develop and provide an analysis service for Service operator 204. AI provider A server 244 implements Artificial Intelligence (AI) analysis algorithm A 242 and is used to analyze health data of a patient using a particular AI algorithm. Similarly, AI provider B server 250 implements AI analysis algorithm B and is used to analyze health data of a patient using a different AI algorithm. It should be noted that although AI provider A 246 and AI provider B 252 are described as implementing AI algorithms, other types of algorithms may be used as well to analyze health data of a patient.

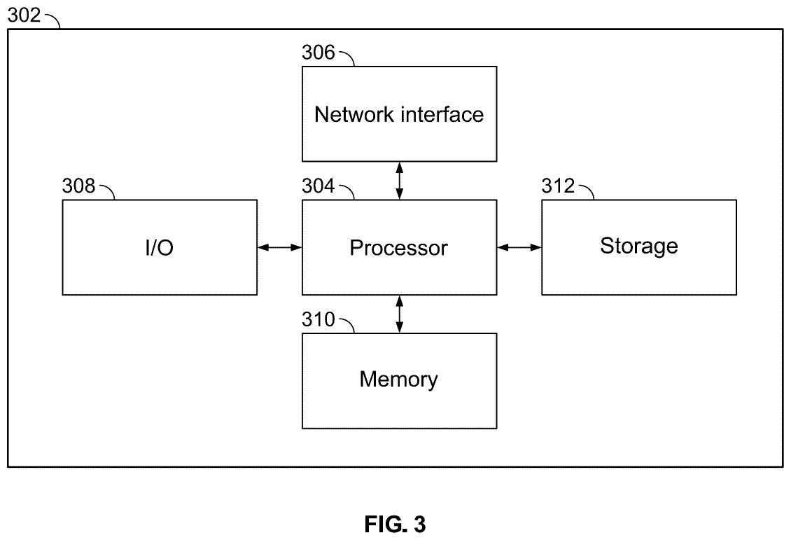

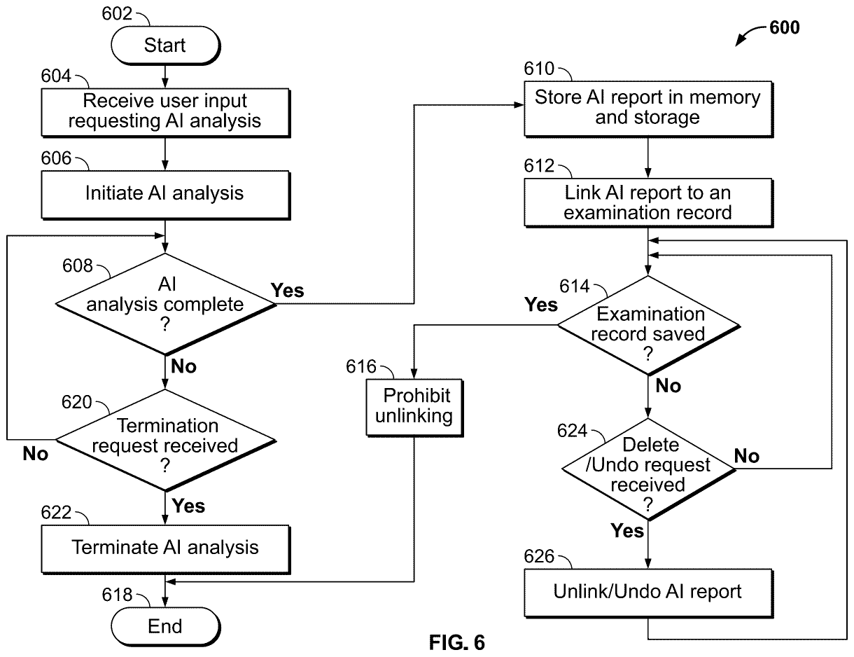

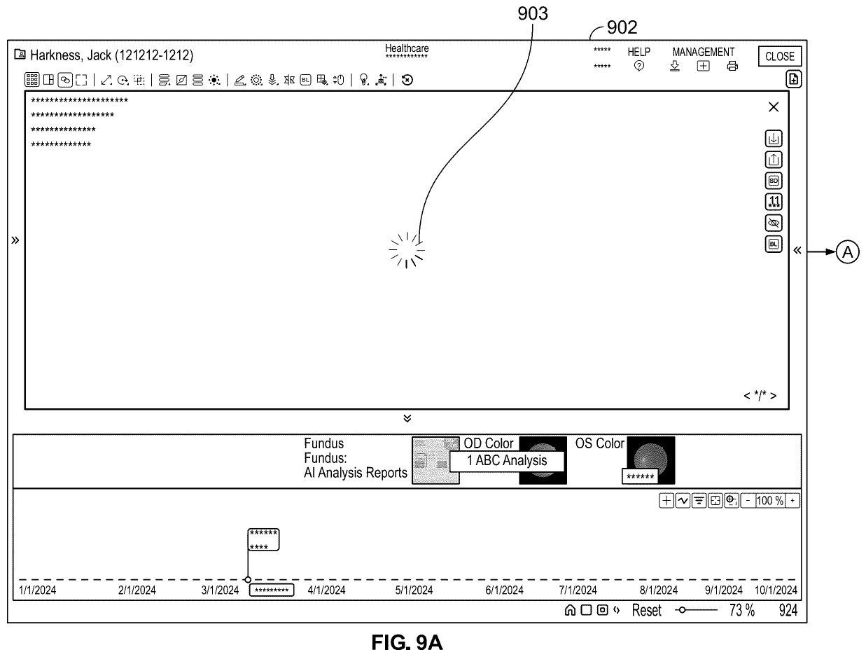

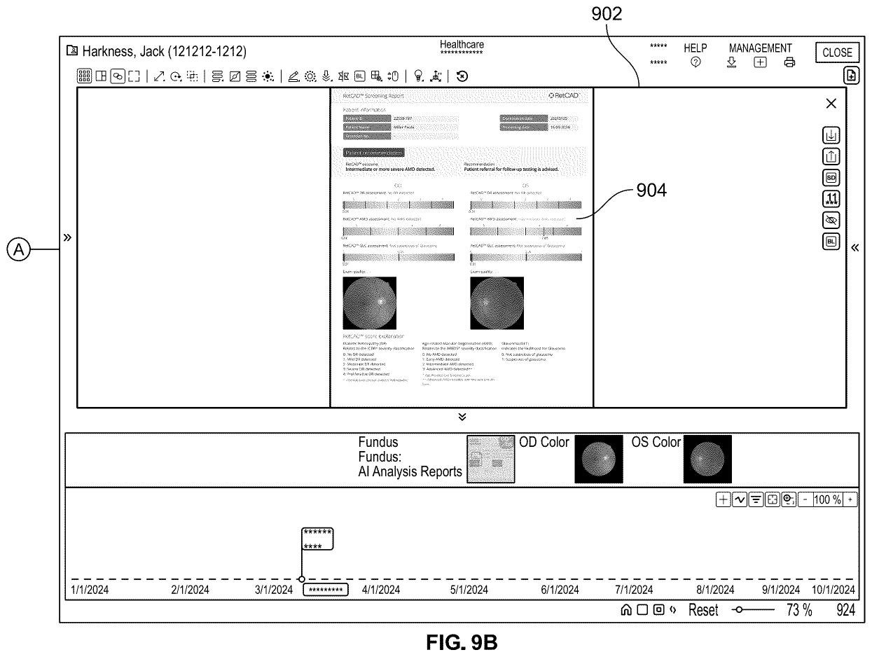

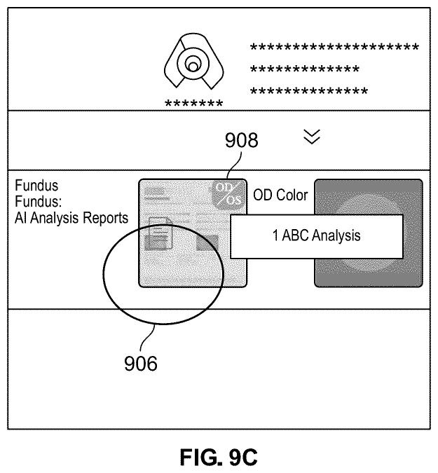

CDSS 100 shown in FIG. 1, as well as other devices described herein, can be implemented using one or more computers. In addition, the hardware identified in FIG. 2 by a bold solid line border as shown in legend 201 of FIG. 2 can also be implemented using one or more computers. A high-level block diagram of such a computer is illustrated in FIG. 3. Computer 302 contains a processor 804 which controls the overall operation of the computer 302 by executing computer program instructions which define such operation. The computer program instructions may be stored in a storage device 312, or other computer readable medium (e.g., magnetic disk, CD ROM, etc.), and loaded into memory 310 when execution of the computer program instructions is desired. Thus, the method steps of FIGS. 6 and 9, the software functions (see legend 201) of FIG. 2, as well as other methods and algorithms described herein, can be defined by the computer program instructions stored in the memory 310 and/or storage 312 and controlled by the processor 804 executing the computer program instructions. For example, the computer program instructions can be implemented as computer executable code programmed by one skilled in the art. Accordingly, by executing the computer program instructions, the processor 304 executes an algorithm defined by the method steps of FIGS. 6 and 9, the software functions of FIG. 2, or other methods and algorithms described herein. The computer 302 also includes one or more network interfaces 306 for communicating with other devices via a network. The computer 302 also includes input/output devices 308 that enable user interaction with the computer 302 (e.g., display, keyboard, mouse, speakers, buttons, etc.) One skilled in the art will recognize that an implementation of an actual computer could contain other components as well, and that FIG. 3 is a high-level representation of some of the components of such a computer for illustrative purposes.

In one embodiment, CDSS 100 is part of data management software 206 and CDSS is opened (i.e., launched) from data management software 206. CDSS 100 is typically launched when a user meets a patient who qualifies for a glaucoma screening. In one embodiment, a patient qualifies if, for example, they show symptoms or if the screening is part of a routine health assessment. The user then checks (e.g., reviews) the risk score of an algorithm that is currently active (i.e., selected). Risk scores of other algorithms can be checked as well. A user can then check examination data (e.g., examination parameters) included in the calculation of risk scores. A user can change or exclude data where necessary and check the risk scores again.

In one embodiment, CDSS 100 can't be launched before the user is logged into data management software 206. The user must also have a patient's information to open a clinical viewer, CDSS 100 must be enabled and the user must have a valid license for the CDSS. A user can then launch CDSS 100 by selecting the glaucoma tab in data management software 206. The dashboard launches and automatically shows a risk score for the patient immediately, calculated for the active algorithm using the latest examination results. In one embodiment, the active algorithm can be selected based on user or organizational preferences.

In one embodiment, CDSS 100 must have at least one algorithm enabled. Depending on configuration, several algorithms may be enabled and available for risk calculations. The active algorithm is the one currently selected in an algorithm view. The other algorithms with licenses are shown as individual tabs in one embodiment. They are referred to as the enabled algorithms.

If more than one algorithm is enabled, a user can change any one of the enabled algorithms to be the active algorithm and view the associated risk score. In one embodiment, each algorithm uses a set of different risk factors to calculate the risk score.

In one embodiment, for each active algorithm, an associated risk score is presented in numerical format. The risk score is also visualized with a gauge where different color risk levels aid a user in reviewing the risk estimation. In one embodiment, individual risk factors associated with a risk score are displayed in addition to a risk score.

In one embodiment, CDSS 100 will automatically select the latest available data to be used in risk calculations. A user can select examinations and/or examination parameters from a drop-down menu that shows all the available examinations within a time range. In one embodiment, to change the data used in the risk calculations a user can select risk factors for a selected eye (i.e., an eye of a patient that has been examined) and the risk score value and the gauge graphic are updated based on the selected risk factors. It should be noted that, in one embodiment, fundus and OCT are interconnected. Changing the fundus image will also change the OCT, and vice versa. If the active algorithm is changed after changing the examination data, the change of examination data is carried over to the new algorithm.

It should be noted that bad or incorrect data can be worse than no data at all. Examinations and/or examination parameters can be left out if a user does not want to include them in the risk score calculation. In one embodiment, the risk score is recalculated after the user selects examinations and/or examination parameters that should be left out of the risk score calculation. If there is not enough data to calculate a risk score after examinations and/or examination parameters are excluded, a risk score will not be calculated.

In one embodiment, CDSS 100 requires the use of discrete data storage (DDS) to safely and securely store data.

The benefits of CDSS 100 include better quality and efficiency of glaucoma testing and services, possible reimbursement for additional testing due to CDSS result indicating medical necessity, reduced medical liability, better efficiency of decision making, better quality of referrals, reduction of over-referrals, and higher patient retention rate.

In one embodiment, AI can be used by opticians performing eye health exams and assist the optician in convincing a patient to agree to further examinations based on the results of the patient's eye health exam. FIG. 4 shows a method 400 for opticians to use AI in order to assist in performing a patient's eye health exam. At step 402, fundus images are generated by examination equipment. At step 404, automatic image analysis is performed using AI. If the AI analysis results show no indication of retinal pathology, then the method proceeds to step 406 where the normal vision exam process continues. If the AI analysis results show an indication of retinal pathology, the method proceeds to step 408 and an additional eye health examination package is offered to the patient. For example, package 410 including exams pertaining to fundus, IOP, visual acuity, and remote reading in various tests (e.g., refraction, visual field, color vision, fundus examination, fundus three-dimensional image analysis (OCT), corneal shape, intracorneal capsular examination, deep vision, contrast examination, slit-lamp microscopy, intraocular pressure test, etc.) may be offered to the patient. Extended package 412 includes exams pertaining to AMD/glaucoma/other indicated pathology, OCT, visual field, IOP, visual acuity, and remote reading in various tests may also be offered to the patient. Examination parameters/data and an optician's tentative diagnosis are the sent to a reading center as shown in step 414 where an ophthalmologist reviews the examination parameters/data and other patient information. The ophthalmologist can reply with a diagnosis and recommendations which, at step 416, the optician can review and perform follow up examinations and/or actions. The ophthalmologist can alternatively reply with a referral to another public or private eye care provider (e.g., another ophthalmologist) as shown in step 418 along with a diagnosis.

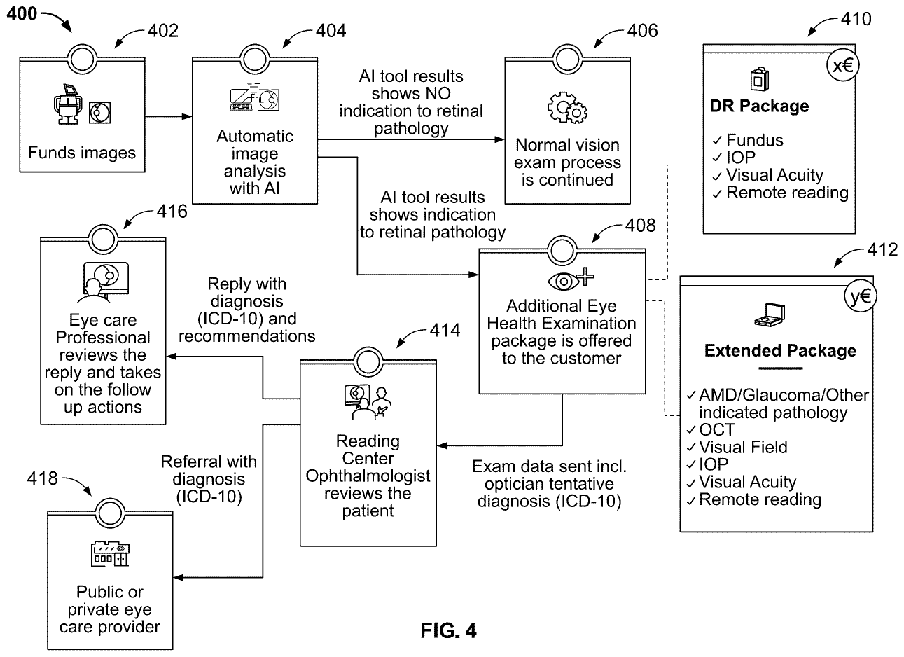

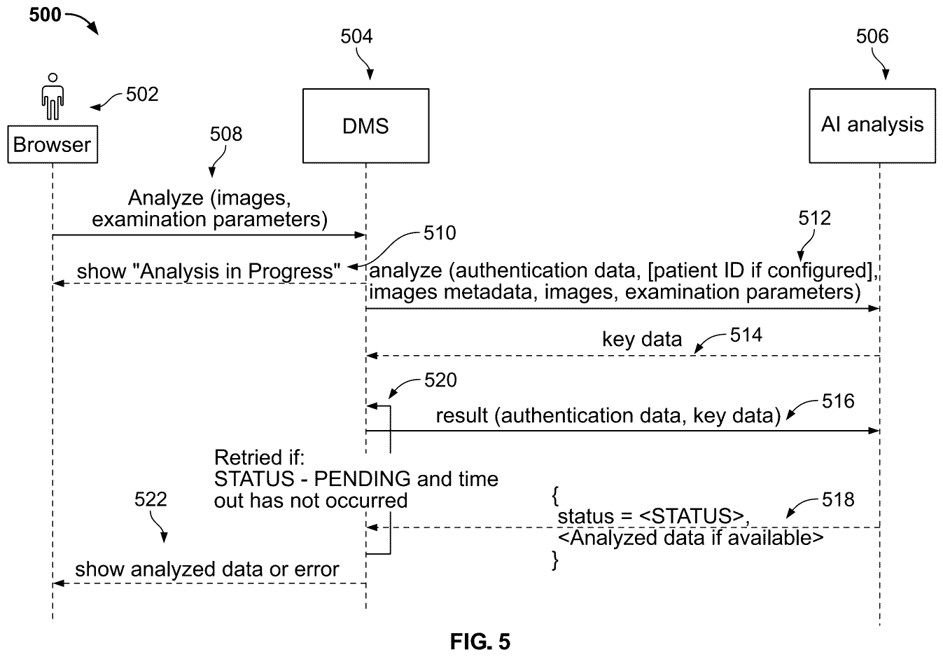

FIG. 5 shows data flow 500 for image analysis indicating how data flows among browser 502, data management software 504 (shown as DMS in FIG. 5), and AI analysis 506 (e.g., AI that is used to analyze images and/or examination data). FIG. 5 also shows session management between data management software 504 and AI analysis 506. It is noted that in FIG. 5, browser 502 is used by a user of data management software 504, and, in one embodiment, the browser can be replaced with other terminal software configured to communicate data management software 504. The vertical axis of the data flow represents the progression of time with the earliest time located at the top of data flow 500 and time progressing downward. The horizontal dashed lines in FIG. 5 represent responses and the horizontal solid lines in FIG. 5 represent data transmission prior to related responses.

At step 508, a user selects a set of images and examination parameters for AI analysis and requests an analysis through browser 502 for data management software 504. In response, at step 510, data management software 504 transmits an indication to display “Processing. . . ” on browser 502 to present to the user. At step 512, data management software 504 transmits an analysis request to AI analysis 506 to analyze the selected data, triggered by the user request of step 508. The request at step 512 includes authentication data, patient information such as a patient identifier (patient ID) (if data management software 504 is configured to send patient information), image metadata, images, and examination parameters. At step 514, in response to step 512, AI analysis 506 returns key data to data management software 504. At step 516, data management software 504 transmits a request of an analysis result, which requests AI analysis 506 to send an AI report produced from the data provided with the analysis request at step 512, including the authentication data of step 512 and the key data of step 514. Including these authentication data and key data serves to verify that AI analysis 506, which is located remotely from data management software 504, has responded in steps 512 and 514. This process is commonly referred to as Challenge-Response Authentication or Session-based Authentication. At step 518, in response to receipt of the result of step 516, AI product 506 transmits the status of its operation and the data that results from the analysis if available. At step 520, if the status is PENDING, indicating that AI product 506 is still analyzing and requesting the system to wait, and the predefined timeout period has not elapsed, data management software 504 will resend the same request sent at step 516. At step 522, the result data of step 518, which will constitute an AI report, is t ransmitted to browser 502, or an error message if there was a problem with the analysis. Browser 502 shows either the AI report or the error message accordingly.

AI can be used to analyze a fundus image to recognize diseases such as diabetic retinopathy (DR), age related macular degeneration (AMD), and glaucoma, and to generate a severity classification. AI can also be used to analyze an OCT scan to recognize pathologies and generate an indication of their severity. AI can further be used for multi-modal analysis of images and scans, other eye data such as perimetry or intraocular pressure (IOP), and a patient's account of their medical history (i.e., anamnesis). AI can also be used for multi-source analysis and forecasting based on various exam data and history, patient history, normative data, different AIs, etc. AI can produce outcomes including diagnosis, recommendations, and clinical guidance. AI analysis can help opticians and optometrists make informed decisions about which patients to send to an ophthalmologist for review, assist in early detection of diseases, and help both optometrists and ophthalmologists provide more services to patients.

In one embodiment, AI is used in screening as follows. A patient is registered (e.g., an electronic medical record for the patient is generated in practice management software). Data management software 206 identifies the electronic medical record of the patient and creates worklists. Examinations are performed according to the worklists and the examination parameters generated during the examinations are stored in data management software 206. In one embodiment, an examination comprises medical test results (e.g., examination parameters) of medical test conducted approximately on the same date. For example, an examination can comprise a plurality of medical test results generated using different techniques and equipment. All of the medical test results generated on approximately the same day are grouped into a single examination. Images are sent for AI based image analysis (e.g., sent to AI provider A 246 and/or AI provider B 252 for analysis). In one embodiment, AI reports generated using medical test results associated with a certain date can be linked to an examination. The examination parameters and AI analysis are reviewed at a screening center. Based on the review, a patient may be referred to an ophthalmologist at a reading center. The ophthalmologist at the reading center reviews patient data including the examination parameters and AI analysis and replies to the screening center if needed. The ophthalmologist at the reading center can also refer the patient to an eye care provider. A referral, if needed is sent to the eye care provider along with relevant patient data. The screening center receives diagnosis, recommendations, and follow up instructions.

Images can be sent for analysis in one of two ways. First, images can be sent for analysis on demand. In this case, a user can select the images to be sent and then cause the selected images to be sent for analysis. Second, a set of rules can be configured to automatically send images for analysis based on one or more factors such as the type of device used to generate the images, etc. In either case, when an AI analysis is complete, the user is notified. In one embodiment, a user may be shown results of the AI analysis. A report may also be generated including the results of the AI analysis. Regulatory information can be included in analysis results and/or reports for convenience. In one embodiment, AI usage is monitored and reports regarding AI usage can be generated for review.

In one embodiment, medical test results are input to an AI function for analysis to generate a report. A display shows information related to the medical test results, data associated with the medical test results, and information associated with report.

It should be noted that the use of AI functions often incurs cost. Not only does executing AI functions require computing resources, but AI functions are often provided as paid services to recover costs associated with software licensing fee, training data fee, and development expenses. In particular, when AI functions are developed by third-party entities, they may be subject to charge. For example, an AI provider that performs analysis of examination parameters using an AI function very often charges for that analysis and associated report. Various fee structures are available, including a flat-rate fee and a usage-based fee. In a usage-based fee, also known as pay-per-use, the AI provider may charge for the entire analysis even if the analysis is canceled before it is finished. In some cases, the AI provider may also charge for duplicate analysis even if the analysis is initiated with the same examination parameters that has been previously analyzed by the same AI function. Accordingly, with respect to those cases, it can be beneficial to prevent termination of an AI analysis once it has begun. It can also be beneficial to prevent the same examination parameters from being analyzed more than once by the same AI function to avoid generation of a duplicate report.

FIG. 6 shows a flowchart of method 600 for generating an AI report using an AI function. In one embodiment, method 600 is performed by data management software 206 shown in FIG. 2. Method 600 begins at start 602 and proceeds to step 604 where user input is received requesting AI analysis. After user input is received at step 604, the method proceeds to step 606 where the AI analysis is initiated. At step 608, it is determined whether AI analysis is complete. If AI analysis is complete, the method proceeds to step 610 where the AI report is stored in both memory and storage (e.g., memory 310 and storage 312 shown in FIG. 3) parallelly for the data persistence. In one embodiment, the AI report can be stored in the AI provider's premises storage, and/or in a third-party storage to maintain redundancy for medical and billing data. Storing the data in multiple storage devices allows the system to ensure data protection. In one embodiment, the AI report can be stored in a blockchain as a hashed value to prevent the deletion and tampering of the medical records. The method then proceeds to step 612 where the AI report is tied to an examination record of a user. That is, the examination record will contain a link, which allow users to call and display the linked AI report to display. The link can be used to open and manipulate the linked AI report. In one embodiment, the link is created and displayed in the examination record as a thumbnail image or an icon image representing the AI report, which is distinct from those representing the examination parameters. In one embodiment, linking is associating various data with other data. For example, all data pertaining to a particular patient can be linked to that patient. The examination record can contain multiple links that are each associated with an analysis result including an AI report. The method then proceeds to step 614, where it is determined whether a user has selected to save the examination record. If the examination has been selected to be saved, the method proceeds to step 616 where unlinking is prohibited and then to step 618 where the method ends. In one embodiment, unlinking comprises disassociating data with other data. For example, when an AI report is unlinked from an examination, the AI report is no longer associated with the examination in a memory or database. The effect of unlinking is to allow the AI report to be invisible on the user interface (UI) from a user's convenience perspective, while ensuring that a record representing the AI report remains recorded in the database or storage for the purposes of medical record retention and billing information management. In one embodiment, the prohibiting comprises deactivating a delete AI report icon.

Returning to step 608, if it is determined that AI analysis is not complete, the method proceeds to step 620 where it is determined whether a termination request has been received. A user can request termination of the AI analysis before AI analysis is complete. If it is determined at step 620 that a termination request has not been received, the method returns to step 608. If it is determined that a termination request has been received at step 620, the method proceeds to step 622 where the AI analysis is terminated and the method proceeds to step 618 where it ends.

Returning to step 614, if it is determined that the examination has not been saved, the method proceeds to step 624 where it is determined whether a delete request has been received. In one embodiment, a delete request is a request input by a user. If a delete request has not been received, the method returns to step 614. If it is determined that a delete request has been received at step 624, the method proceeds to step 626 where the AI report is unlinked from the examination and the method proceeds to step 618 where it ends.

In one embodiment, a same set of examination parameters that were used to generate a previous AI report using a particular AI function are prohibited from being used to generate the previous AI report again using the particular AI function. This is to prevent duplicate charges caused by the user inadvertently performing the same analysis. In another embodiment, the same set of examination parameters are prohibited from being used by disabling selection of the same set of examination parameters that have been previously used to generate the previous AI report. In yet another embodiment, the same set of examination parameters are prohibited from being used by disabling selection of the particular AI function used to generate the previous AI report when the same set of examination parameters previously selected to generate the AI report are selected. In one embodiment, examination parameter icons representing the same set of examination parameters that have been previously used to generate the previous AI report each have an identifier indicating that that examination parameter was previously used with the particular AI function to generate the previous AI report.

In one embodiment, an AI function to generate the AI report is automatically selected based on the examination parameters to be analyzed. In one embodiment, the AI function is automatically selected further based on a service operator identifier. In one embodiment, the AI function is automatically selected further based on an apparatus identifier and the apparatus identifier can specify a machine learning model to analyze the examination parameters. In one embodiment, the apparatus identifier identifies an apparatus that was used to generate data to train the machine learning model.

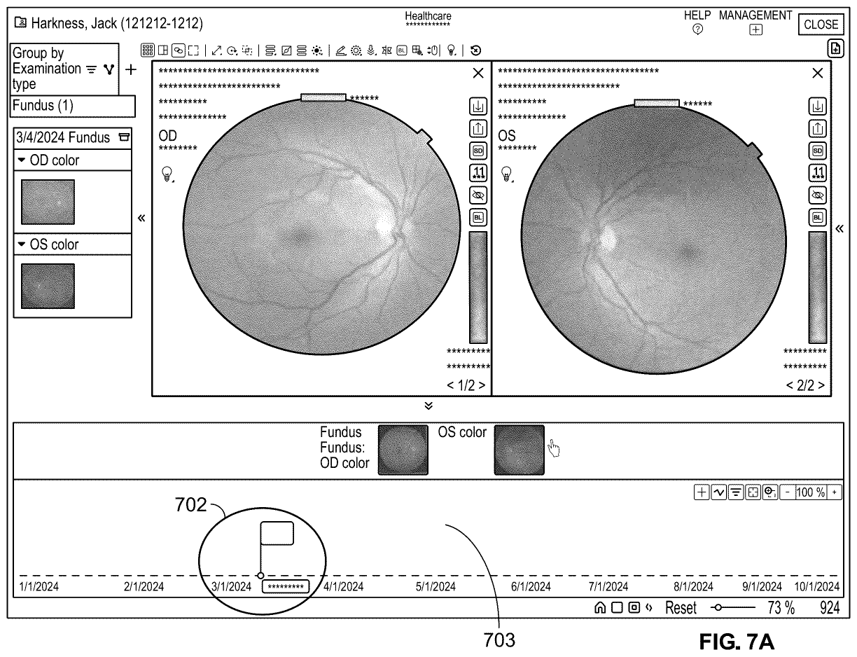

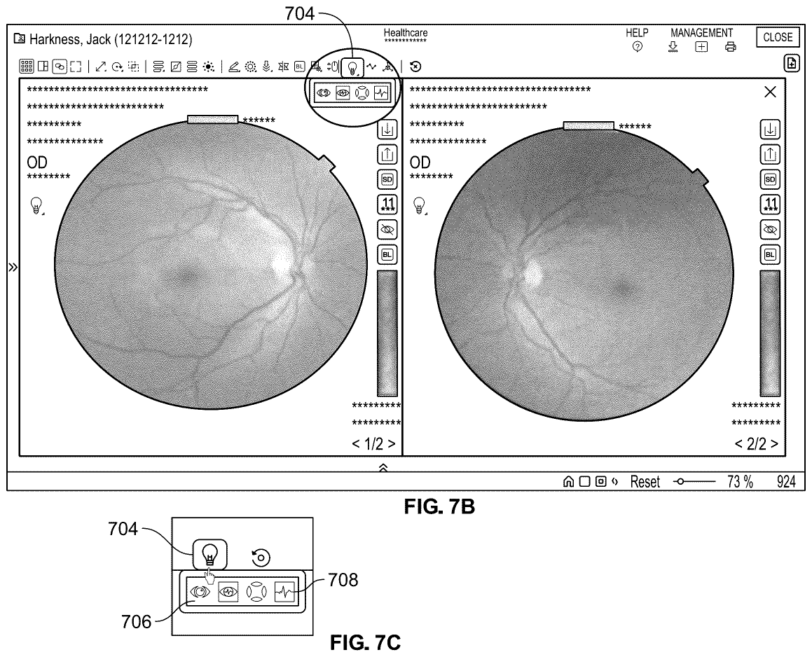

In one embodiment, a user, such as an eye care provider, can choose the examination parameters to be analyzed by an AI function by a third-party AI provider for health check and screening purposes. In one embodiment, this selection occurs during step 604 shown in FIG. 6. FIGS. 7A-7C shows a screen flow of a user selecting examination parameters on a timeline for analysis by an AI function. FIG. 7A shows examination parameters icon 702 located on timeline 703. Examination parameters icon 702 has been selected by a user. FIG. 7B shows AI analysis icon 704 with an on-screen pointer (e.g., a mouse pointer) hovering over it. In response to the hovering over AI analysis icon 704, AI function icons 706 and 708 are displayed as shown in FIG. 7C. Each of AI function icons 706 and 708 identify AI functions that can be selected by clicking on one of them to request analysis of examination parameters associated with examination parameters icon 702.

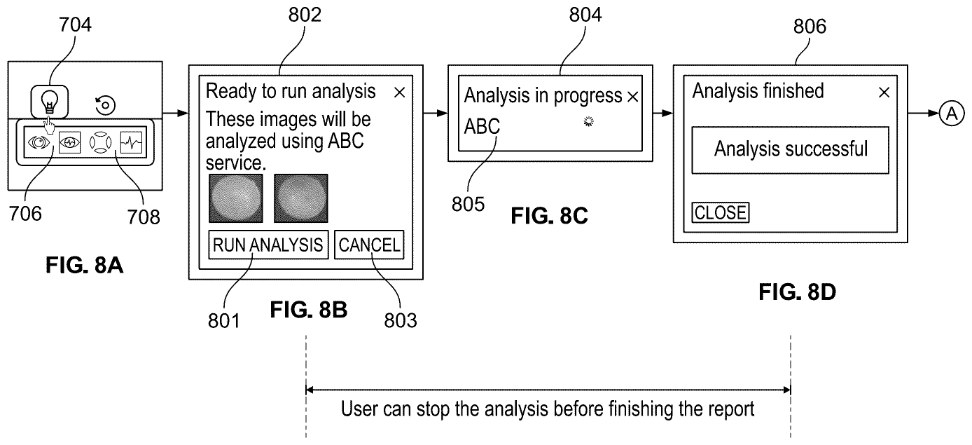

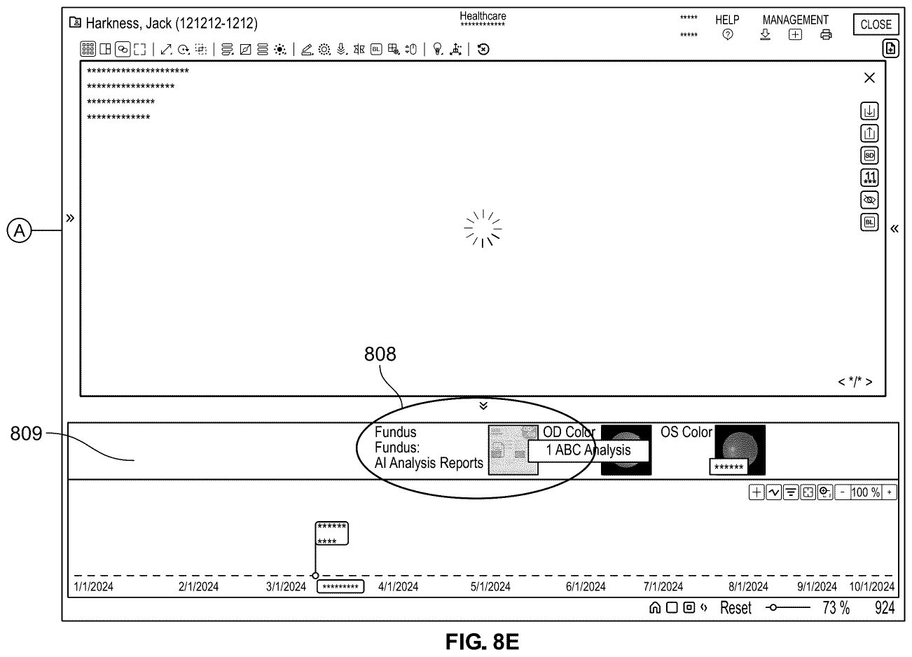

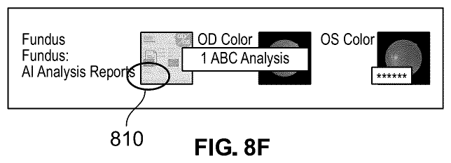

FIGS. 8A-8F show a screen flow of a user selecting an AI function to analyze examination data. FIG. 8A shows a user hovering over AI analysis icon 704 to select an AI function, represented by one of AI function icons 706 and 708, that will be used to analyze examination parameters. In one embodiment, selection of an AI function can occur in step 604 shown in FIG. 6. FIG. 8B shows pop-up window 802 which is displayed in response to a user selecting an AI function, in this case, the ABC AI function, to analyze examination parameters. Pop-up window 802 shows the user the examination parameters that will be analyzed by the selected AI function (i.e., the AI function ABC) and displays a “Run Analysis” button 801 that a user can select to run the AI analysis and a “Cancel” button 803 that a user can select to cancel the analysis. In one embodiment, selection of the “Run Analysis” button 801 initiates AI analysis as described in connection with step 606 shown in FIG. 6. FIG. 8C shows pop-up window 804 which appears in response to the user selecting to run the analysis. Pop-up window 804 shows text indicating that the analysis is in progress. In one embodiment, progress of the analysis can be shown using a progress bar or loading indicator (not shown). AI function identifier 805 indicates the AI function being used to analyze examination data can also be displayed in pop-up window 804. FIG. 8D shows pop-up window 806 that is displayed to a user after the AI analysis has finished. In one embodiment, pop-up window 806 displays text indicating the AI analysis was successful and that the report is available for viewing. FIG. 8E shows report icon 808 which is a thumbnail of the report in thumbnail viewing area 809. FIG. 8F shows report icon 808 having delete icon 810 that can be selected by a user to unlink the report from a patient's timeline and the thumbnail viewing area (e.g., unlink from an examination). It should be noted that a user can request to stop the analysis at any time between the display of pop-up window 802, shown in FIG. 8B, until the display of pop-up window 806, shown in FIG. 8D.

FIGS. 9A-9C show a screen flow of generation and display of a report (e.g. the report generated in FIGS. 8A-8F) after a user has selected the examination parameters and AI function to analyze the selected parameters. FIG. 9A shows display 902 while a report is being prepared for display as indicated by loading icon 903. FIG. 9B shows display 902 displaying report 904 while FIG. 9C shows a thumbnail 908 of report having an icon 906 that is displayed when an on-screen pointer (e.g., a pointing device such as a mouse) is hovered over thumbnail 908. Icon 906 can be selected by a user to request that the report be deleted (however, as described herein, the report may not be actually deleted).

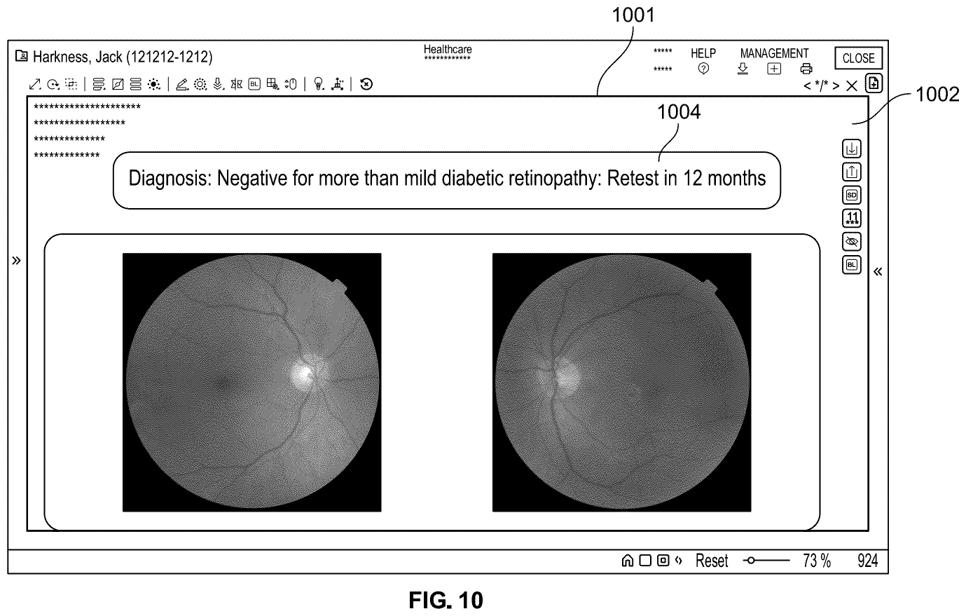

FIG. 10 shows display 1001 including report 1002 that is generated by an AI function (e.g., reports generated as described in connection with FIGS. 8A-8F and 9A-9C) and displayed to a user. In one embodiment, report 1002 includes diagnosis 1004. Some diagnoses are simple because those diagnoses are used for primary care physicians who are not eye care specialists. Users and patients can review report 1002 and discuss whether further analysis is needed. Other diagnoses may contain more detailed information for display to an eye care specialist.

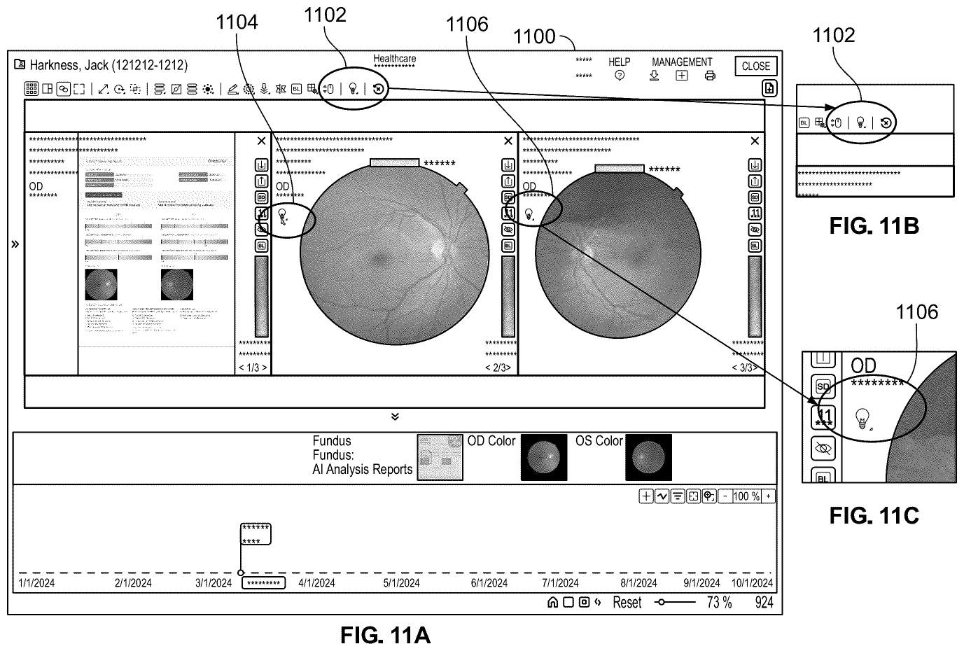

FIGS. 11A-11C shows how a user is restricted from generating a report using the same examination parameters and AI function as those used to create a previous report. FIG. 11A shows display 1100 in which AI function icon 1102 (shown in detailed in FIG. 11B) is greyed out indicating that it cannot be selected. Also shown in display 1100 is icon 1104 and 1106 (shown in detail in FIG. 11C) which indicate that the associated examination data shown has already been used to generate a report using the AI function identified by AI function icon 1102. In one embodiment, AI function icon 1102 is greyed out because it was previously used to analyze the currently selected examination parameters.

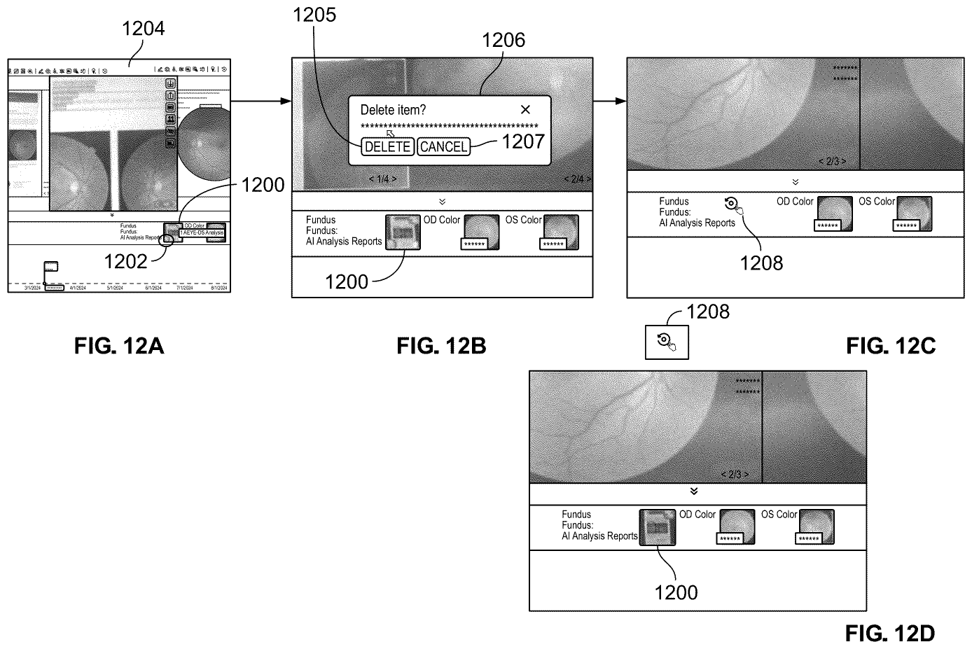

FIGS. 12A-12D show screen flows showing how a user can request to delete a report. FIG. 12A shows delete button 1202 located on a report thumbnail 1200 in display 1204. FIG. 12B shows pop-up window 1206 which is displayed in response to a user selecting delete button 1202. Pop-up window 1206 displays text asking if a user wants to delete a report and provides delete button 1205 and cancel button 1207. If a user selects delete button 1205, the report is unlinked (but not actually deleted and since the system prohibits the report file to be erased once it is produced and stored and maintains the report physically stored) from an examination record associated with a patient record. If a user selects cancel button 1207, the report remains linked to the examination record associated with the patient record. FIG. 12C shows undo button 1208 (also shown in detail below FIG. 12C) which allows a user to undo the delete of the report. FIG. 12D shows report thumbnail 1200 displayed after a user selects undo button 1208. A user may select undo button 1208 if the user changed their mind regarding whether a report should be linked to an examination. It should be noted that “delete” in this embodiment refers to unlinking of the report from an examination and this unlinking is described in detail below. The report is still stored (or maintained) in memory and storage despite the request to “delete” in this embodiment.

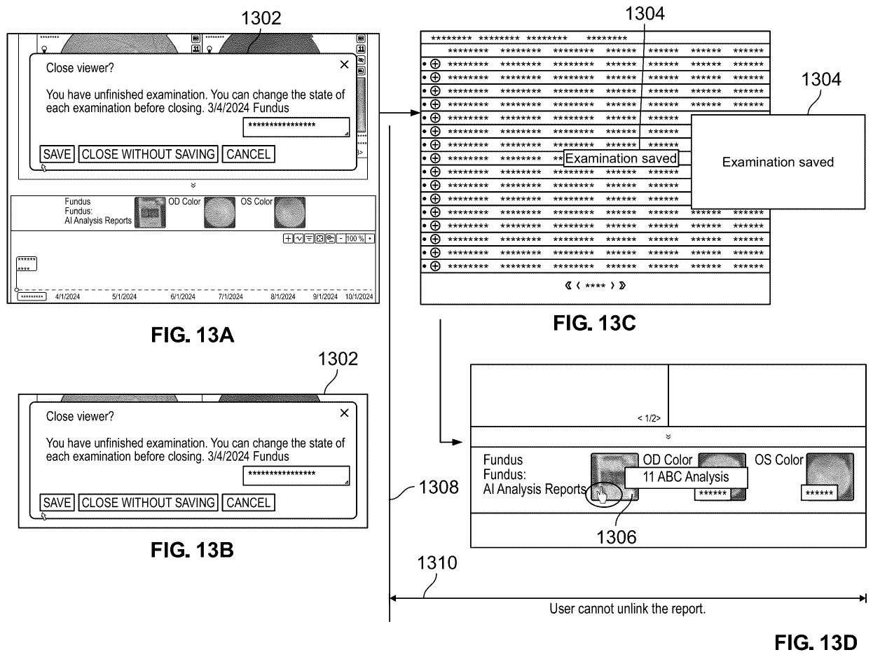

FIGS. 13A-13D show screen flows pertaining to how a user can delete an AI report before an examination is finished (i.e., before the examination is saved). FIG. 13A shows pop-up window 1302 which is displayed to a user in response to the user selecting to close a viewer (e.g., a patient data viewer). FIG. 13B shows a detail of pop-up window 1302 which provides a user with options to save an examination, close without saving the examination, and canceling the closing operation. If the user chooses to save the examination, pop-up window 1304 is displayed with the text “Examination saved” indicating that the examination has been saved. AI report thumbnail 1306 is displayed without an option to delete the AI report because the examination including the AI report has been saved with a copy of the AI report. After a user selects to save an examination with an AI report, the AI report can no longer be deleted by the user. Line 1308 is located to the right of FIGS. 13A and 13B and to the left of FIGS. 13C and 13D. Saving the examination record in the system allow users to commit or finalize it, meaning that once the examination is finalized, the AI report associated with that examination can no longer be unlinked whereas unlinking the AI report is permitted prior to saving the examination record, i.e., before finalizing the examination. Line 1308 identifies a cutoff between when a user can unlink a report to an examination (FIGS. 13A and 13B) and when a user cannot unlink a report to an examination (FIGS. 13C and 13D) as shown by line 1310 shown below FIGS. 13C and 13D.

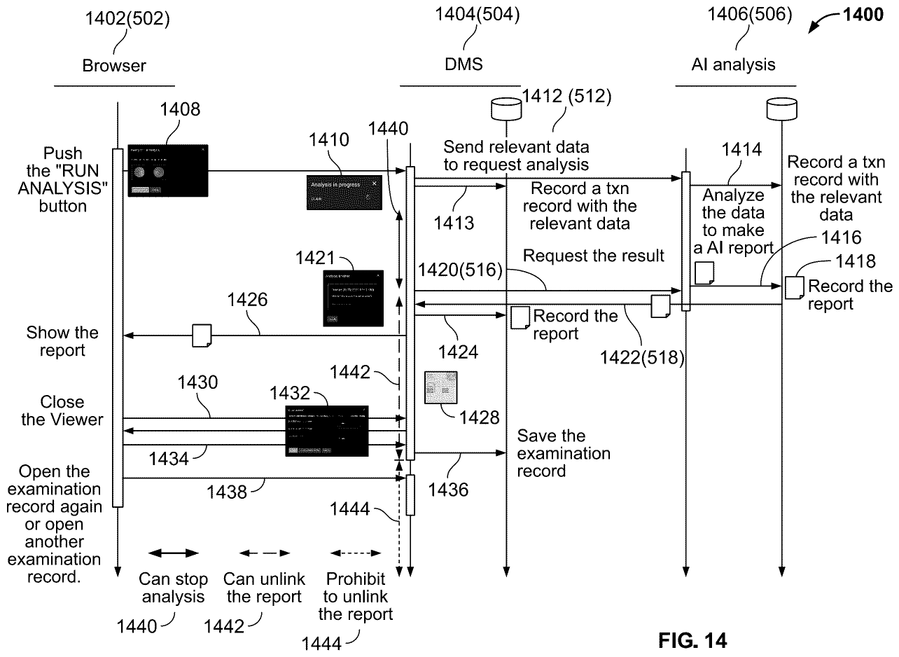

FIG. 14 shows data flow 1400 which indicates data flow among user 1402, data management software 1404, and AI analysis 1406. Depicting the same flow illustrated in FIG. 5, FIG. 14 provides the data flow in a different perspective to specifically illustrate how the system manages and stores AI reports and transaction records. For this purpose, FIG. 14 includes two lines depicted beneath software components such as data management software 1404 and AI analysis 1406, with the two lines respectively representing an application process and a storing process involving memory, database, and storage. The vertical axis of the data flow represents the progression of time with the earliest time located at the top of data flow 1400 and time progressing downward. The sequence begins with a user selecting “Run Analysis” in pop-up window 1408 (corresponding to FIG. 8B). In response, pop-up window 1410 is displayed to the user (corresponding to FIG. 8C) and data management software 1404 sends an analysis request to AI analysis 1406, as shown by step 1412, with relevant data including patient ID, image data, and examination parameters to request the analysis and produce an AI report. Data management software 1404 records a transaction record with examination parameters at 1413. AI analysis 1406 records a transaction record with some of the relevant data at 1414. AI analysis 1406 then analyses the examination parameters using the AI function, as shown by step 1416, to produce an AI report. After the AI report has been successfully produced, AI analysis 1406 records the AI report as indicated by step 1418. Data management software 1404 sends an analysis-result request, as shown by 1420, to request AI analysis to send the result of analysis back and/or to inquire the status of analysis (corresponding to step 516 in FIG. 5), and in response, receives data 1422 including the AI report (corresponding to step 518 in FIG. 5). Data management software 1402 display pop-up window 1421 (corresponding to FIG. 8D) to the user indicating that the AI analysis is finished. In response, data management software 1404 records the AI report locally as shown by step 1424 and also transmits the AI report to browser 1402 for display as shown by step 1426. Browser 1402 then displays AI report 1428 to user 1402 including an icon that allows user 1402 to unlink the AI report from the examination.

After viewing the AI report, user then requests to close the view through browser 1402, as shown by step 1430. In response, data management software 1404 displays pop-up window 1432 to user asking user to confirm that they want to close the viewer. In response to user confirming shown by step 1434, data management software 1404 saves the examination record, as shown by step 1436. Saving examination records indicates the commit and finalization of the examination records, in which user can no longer unlink the AI report from the examination record. The legend at the bottom of FIG. 14 indicates some periods for different managements for records: A solid line 1440 indicates a period when a user can stop analysis, a broken line 1442 does when a user can unlink a report from an examination record, or a dashed line does when the user is prohibited from unlinking the report from an examination record. The lines shown in the legend are also shown vertically to the left to a line beneath data management software 1404 shown in FIG. 14 and indicate when a user can stop analysis 1440, can unlink a report from an examination 1442, or when the user is prohibited from unlinking the report from an examination 1444. In one embodiment, prohibiting comprises deactivating a delete AI report icon.

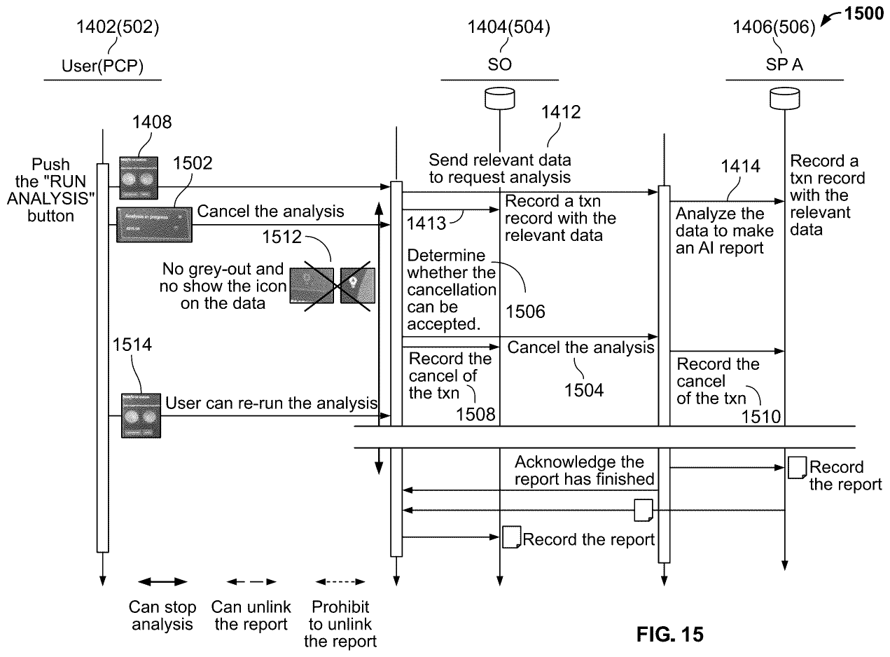

FIG. 15 shows data flow 1500 which indicates data flow among browser 1402, data management software 1404, and AI analysis 1406 when a user chooses to run an AI analysis but then requests to cancel that analysis. Steps 1408, 1412, 1413, and 1414 are the same as shown in FIG. 14, however, user 1402 requests to cancel the analysis at 1502, and data management software 206, upon receiving the cancellation request, determines whether the cancellation request is acceptable, as shown by 1506. In one embodiment, data management software 1404 may accept the cancellation until data management software 1404 issues an analysis-result request, as shown in step 1420, since the analysis-result request is issued when data management software 1404 considers that AI analysis functions have completed the analysis and are ready to send the result back. In one embodiment, data management software 1404 may accept the cancellation request until a predetermined period has elapsed after issuing an analysis request, as shown by step 1412, where the predetermined period is set based on the time required for AI analyses or AI providers to consume a certain amount of their resources that should be billed to service operators. Based on the determination data management software 1404 forwards this cancelation to AI analysis 1406 as shown by step 1504. Triggered by the user's cancelation, data management software 1404 records the cancelation of the transaction at 1508 and AI analysis 1406 records the cancelation of the transaction at 1510. As shown by images 1512, the AI analysis icon is no longer greyed out for this combination of AI function and examination parameters and the examination parameters are displayed without an icon indicating that the examination parameters were already analyzed using a particular AI function. Image 1514 indicates that a user can choose to re-run the analysis that was previously canceled. The cancellation function of analysis is designed to improve user convenience by allowing users to stop an analysis that was accidentally initiated. It is preferable to accept the cancellation if the AI provider's analysis resources have not yet been consumed.

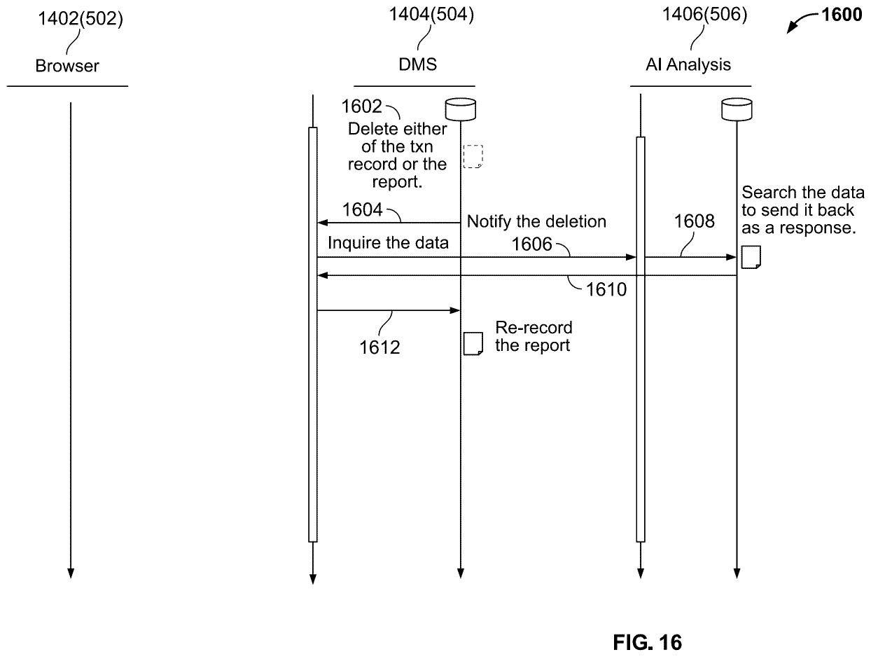

FIG. 16 shows data flow 1600 among data management software1404 and AI analysis 1406 to illustrate processes how AI reports or transaction records are restored when unintentionally deleted from the storage. These data should be stored in a secure manner to fulfill the obligation to retain medical records or to verify and audit the billing details for AI providers. These data should be stored in a redundant manner in both premises of the service operator and the AI provider. When the data is detected in either of the premises, this data flow 1600 restores the deleted data by using the remaining copy stored in the other premises. At 1602, an unintentional deletion occurs in an AI reports or a transaction record stored in the storage or database for data management software 1404. The following description describes an example in which the AI report is deleted. At 1604 notification of the deletion is provided from the storage and database. In one embodiment, data management software 1404 may implement a background service that periodically monitors the existence of transaction records and/or reports and detects any deletion. In another embodiment, a database may implement a trigger function to notify the system whenever a DELETE event occurs for a specified record. Upon the notification of the deletion, data management software 1404 requests AI analysis 1406 to send the AI report that has been stored in AI analysis's premises as shown in FIG. 16 at 1606. At 1608, the request is received and AI analysis 1406 seeks the AI report in their storage and database and return the data. At 1610, the data management software 1404 stores the AI report in their storage and database, locally in their premises. In one embodiment, data management software 1014 may compute the hash value of the AI report and compare it with the previous hash value of the AI report that has recorded before deletion so that the system could verify the data integrity of the deleted AI report. If the hash values do not match, data management software 1014 may be configured to notify a system manager of the service operator.

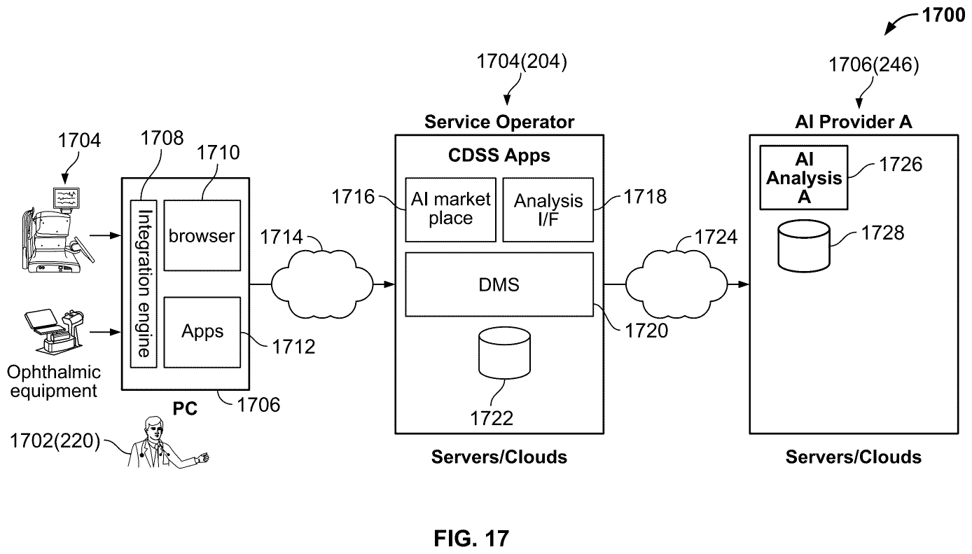

FIG. 17 shows system configuration 1700 according to one embodiment, depicting a typical hardware setup with software components, in which the premises of user 1702, the premises of service operator 1704, and the premises of the AI provider A 1706 are interconnected via public network 1714, 1724. That is, each premises is remotely located and interactively connected. User 1702 interacts with ophthalmic equipment 1704 and personal computer (PC) 1706 which supports integration engine 1708 for interacting with ophthalmic equipment 1704 and interacting with browser 1710 and software applications (apps) 1712 that connect to data management software 1704. (PC 1706 is a simplified illustration of integration server 210, connector 216, and user workstation 224 in FIG. 2). PC 1706 is in communication with service operator 1704 via public network 1714. In one embodiment, service operator 1704 includes AI marketplace 1716, analysis interface 1718, data management software 1720, and database 1722. Service operator 1704 is in communication with AI provider A 1706 via public network 1724. In one embodiment, AI provider A 1706 incudes AI analysis A 1726 and database 1728. It should be noted that in this embodiment, service operator 1704 and AI provider A 1706 are located on different servers in different clouds. This system configuration is the most typical setup used when service operators and AI providers are different entities.

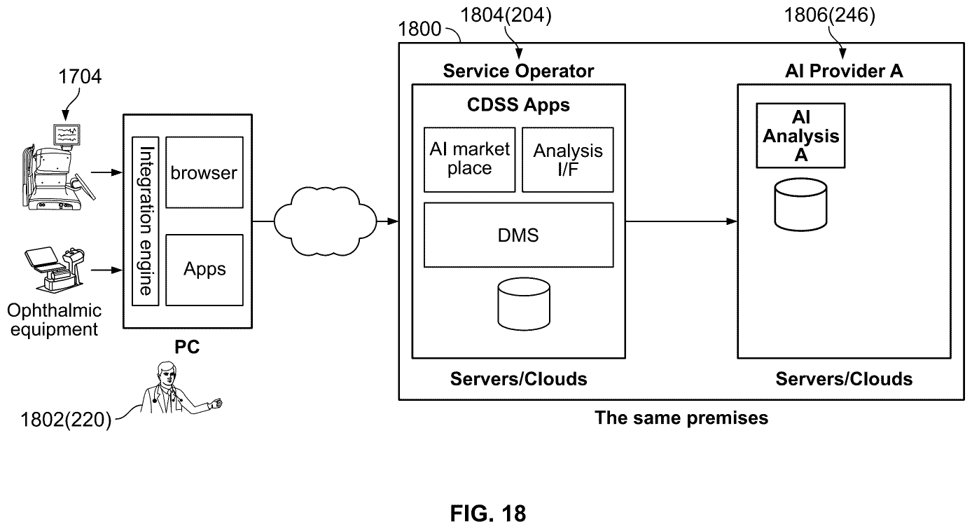

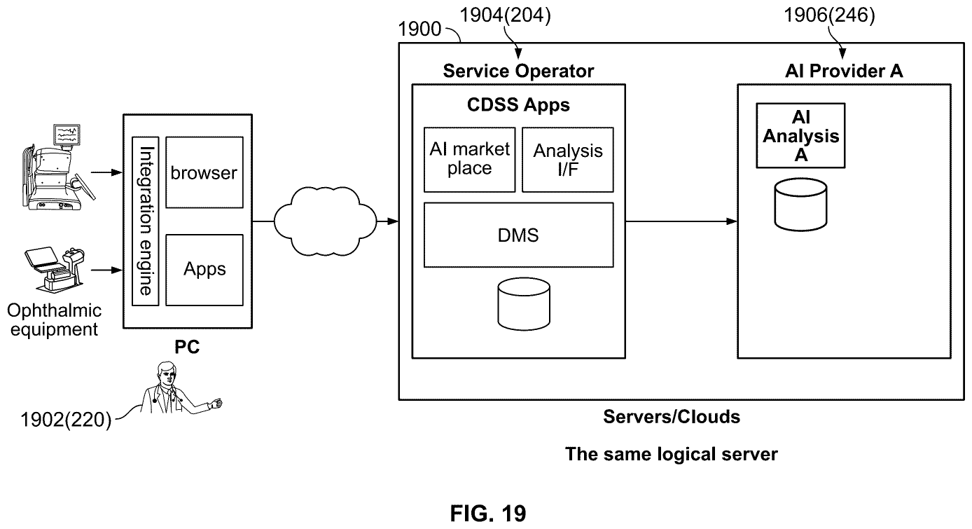

FIG. 18 and FIG. 19 each show a variation of the embodiment shown in FIG. 17. FIG. 18 shows an embodiment in which service operator 1704 and AI provider A 1706 are located at the same premises. Here, “the same premises” refers to a state in which services are provided within the same business entity's service infrastructure, such as within a cloud service environment or the same data center. This embodiment is typically applied when a service operator leases a portion of its cloud service infrastructure to an AI provider.

FIG. 19 shows an embodiment in which service operator 1704 and AI provider A 1706 are located on the same logical server but are logically separated. This embodiment is typically applied when a service operator and an AI provider are operated by closely related business entities.

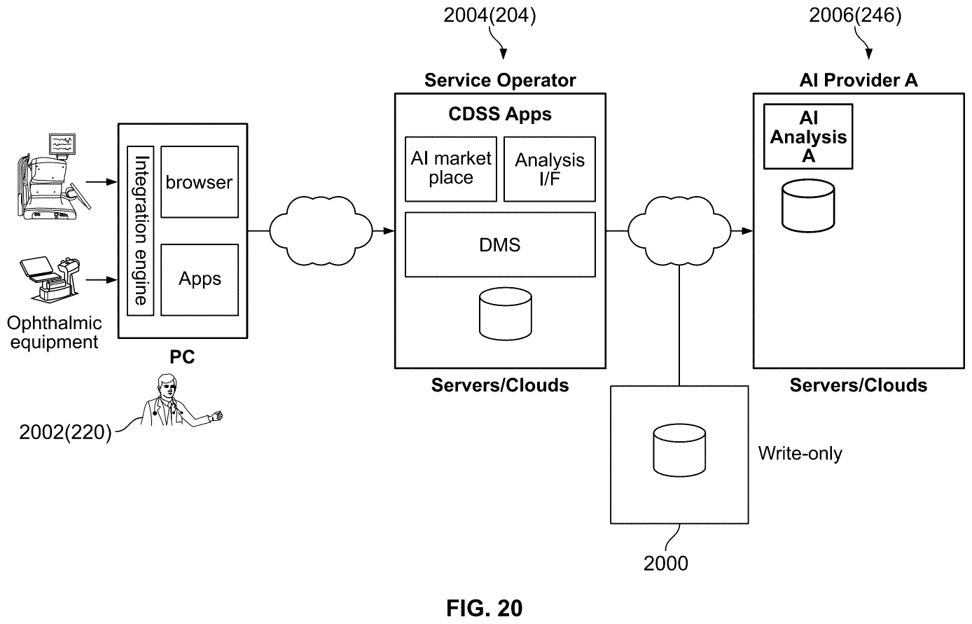

FIG. 20 shows an embodiment in which service operator 1704 and AI provider A 1706 are both in communication with database 2000 which is configured to store examination parameters, AI reports and/or transaction records. By storing data in a redundant database system, data can be restored from the records in database 2000 using a data flow similar to the one described in method 1600, but involving database 2000 instead, when either the service operator's or the AI provider's data is deleted. In one embodiment, database 2000 is write-only and data stored on database 2000 cannot be deleted. This prevents data tampering and accidental data deletion. Although this embodiment is illustrated based on the system configuration of embodiment 1700, it should be noted that it may also be implemented in variations such as embodiments 1800 and 1900.

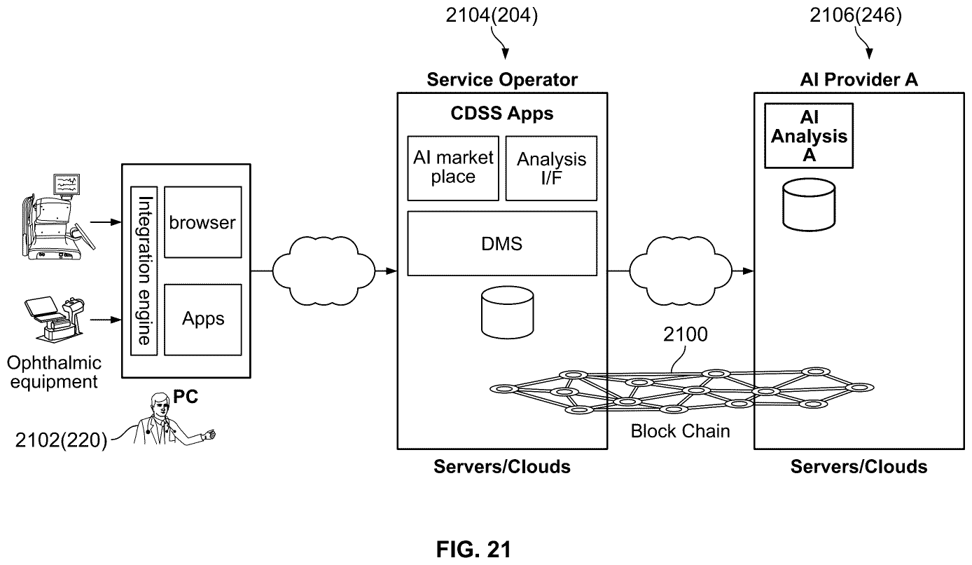

FIG. 21 shows an embodiment in which data, such as examination parameters and reports, are stored on block chain 2100. Blockchain technology is a method for recording and managing data in a decentralized network, characterized by its resistance to tampering and the inability to delete recorded data. Each piece of data is stored as a “block” and linked in a chain using cryptographic techniques. Because modifying a recorded block requires recalculating all related blocks, data tampering is practically infeasible. Additionally, since blockchain adopts an append-only structure, new data can be added without deleting existing data, ensuring data transparency and integrity. Each block in the blockchain contains the recorded data, the hash value of the current block, and the hash value of the previous block. Examination parameters and AI report data can be recorded as a part of blocks. By utilizing blockchain for these records, the reliability and availability of the data are enhanced. Although this embodiment is illustrated based on the system configuration of embodiment 1700, it should be noted that variations such as embodiments 1800 and 1900 may also be implemented.

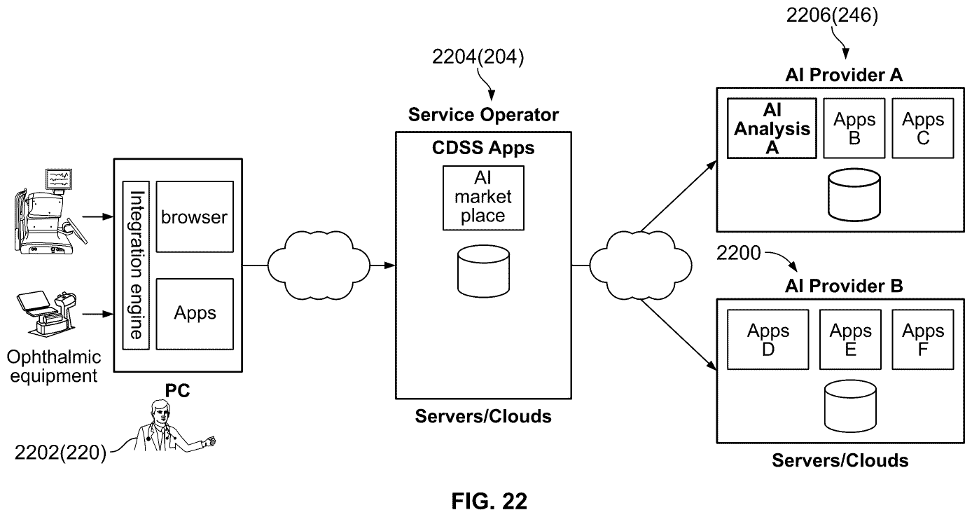

FIG. 22 shows an embodiment in which service operator 1704 is in communication with more than one AI providers. As shown in FIG. 22, service operator 1704 is in communication with service provide A 1706 and AI provider B 2200. Each AI provider can provide AI analysis using different AI functions (e.g., applications).

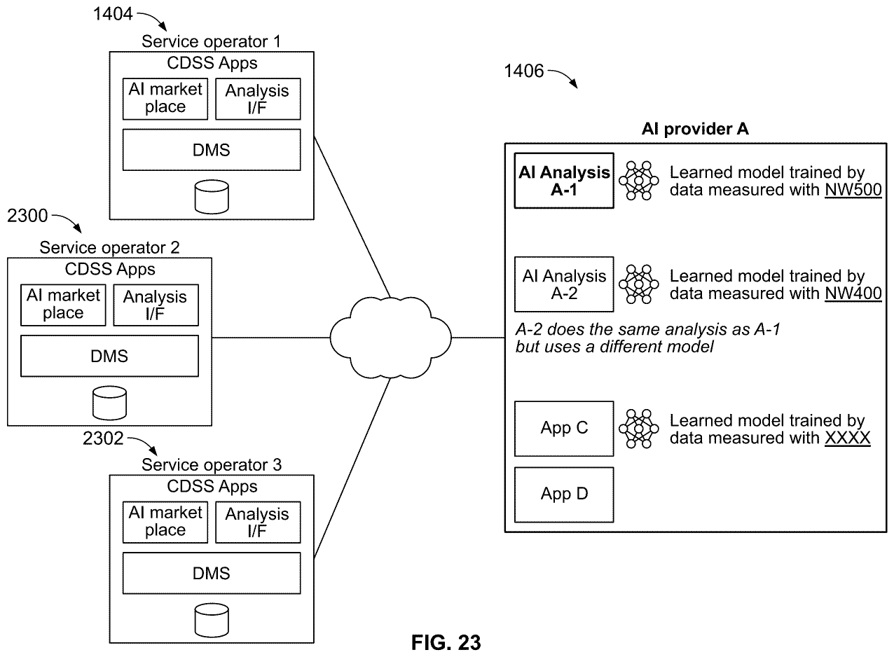

FIG. 23 shows an embodiment in which multiple service operators 1704, 2300, and 2302 are in communication with AI provider A 1706.

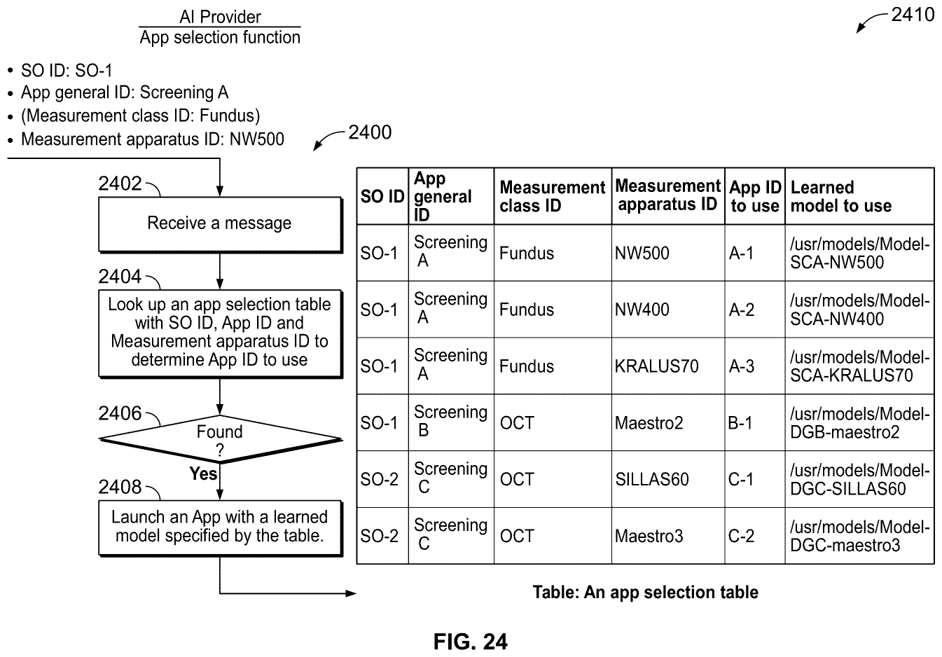

FIG. 24 shows an application selection function according to one embodiment, allowing the selection of an AI analysis function among from multiple options provided by an AI provider. At AI provider A, method 2400 is performed. At step 202, a message is received from a service operator including the following variables: a service operator ID (An ID that uniquely identifies a service provider); an Application ID (An ID that uniquely identifies an application managed by the service provider); measurement class ID (An ID indicating the measurement technology or a specific area of the eye to measure, including OCT (Optical Coherence Tomography), OCTA (OCT Angiography), Fundus Imaging (Fundus Camera), and Fluorescein Fundus Angiography); and a measurement apparatus ID (An ID indicating the model name of the apparatus actually used for the specified measurement class). At step 2404, an application selection table, such as table 2410, is looked up to determine what application ID (An ID that uniquely identifies the AI analysis function within the AI provider) to use and what learned model to use based on the service operator ID, the application ID, the measurement class ID, and the measurement apparatus ID. At step 2406, it is determined whether an application ID and learned model are identified in application table 2410. If an application ID and learned model are identified in application table 2410, the method proceeds to step 2408 wherein the identified application and learned model specified in application table 2410 are used to analyze examination parameters. If an application ID and learned model are not identified in application table 2410 at step 2406, an error message is displayed to a user indicating that no application ID or learned model have been identified. The method 2400, implemented with the application selection table, is necessary because measurement data, particularly OCT-generated images and fundus images, may vary depending on the measurement apparatus, even within the same measurement class. This is useful implementation for a machine leaning scheme in which measurement data is used for training to produce a learned model that classifies, diagnoses, verifies with probability or scores new measurement data. This is particularly important when the service operator manages many opticians and clinics, resulting in data sent by the service operator consisting of measurements taken with multiple medical apparatuses.

The foregoing Detailed Description is to be understood as being in every respect illustrative and exemplary, but not restrictive, and the scope of the inventive concept disclosed herein is not to be determined from the Detailed Description, but rather from the claims as interpreted according to the full breadth permitted by the patent laws. It is to be understood that the embodiments shown and described herein are only illustrative of the principles of the inventive concept and that various modifications may be implemented by those skilled in the art without departing from the scope and spirit of the inventive concept. Those skilled in the art could implement various other feature combinations without departing from the scope and spirit of the inventive concept.

1. A method comprising:
receiving user input requesting AI analysis of examination parameters;
initiating the AI analysis of the examination parameters in response to the receiving user input requesting AI analysis of examination parameters to generate an AI report;
storing the AI report in storage;
linking the AI report with an examination record wherein a link is created to manipulate the AI report; and
in response to receiving a delete request of the AI report before the examination record has been saved, unlinking the AI report from the examination record by deleting the link while maintaining the AI report in storage.

2. The method of claim 1, further comprising:
in response to receiving a terminate request before an AI report has been received, terminating the AI analysis; and
in response to receiving a terminate request after an AI report has been received, prohibiting terminating the AI analysis.

3. The method of claim 1, further comprising:
prohibiting unlinking of the AI report from the examination record after the examination has been saved.

4. The method of claim 3, wherein the prohibiting unlinking comprises deactivating a delete AI report icon.

5. The method of claim 1, wherein the link is represented with a thumbnail image of AI report on which a delete AI report icon is displayed while the AI report icon is being hovered over with by a pointing device.

6. The method of claim 1, further comprising:
prohibiting a same set of examination parameters that were used to generate a previous AI report using a particular AI function from being used to generate the previous AI report again using the particular AI function.

7. The method of claim 5, wherein the same set of examination parameters are prohibited from being used by disabling selection of the same set of examination parameters that have been previously used to generate a previous AI report.

8. The method of claim 6, wherein examination parameter icons representing the same set of examination parameters that have been previously used to generate the previous AI report each have an identifier indicating that that examination parameter was previously used with the particular AI function to generate the previous AI report.

9. The method of claim 5, wherein the same set of examination parameters are prohibited from being used by disabling selection of the particular AI function used to generate a previous AI report when the same set of examination parameters previously selected to generate the AI report are selected.

10. The method of claim 1, wherein an AI function to generate the AI report is automatically selected based on the examination parameters to be analyzed.

11. The method of claim 1, further comprising:
storing the AI report in a storage located at a different premises;
monitoring the deletion of AI reports at both premises; and
restoring the AI report when detecting the deletion of the AI reports in either of the premises by copying the AI report remained in the storage to another.

12. The method of claim 10, further comprising:
storing the AI report in a write-only storage.

13. The method of claim 12, wherein the write-only storage is implemented by using a block chain technology wherein the AI report is appended as a block to existing blocks with the hash value of the previous block.

14. An apparatus comprising:
a processor; and
a memory to store computer program instructions, which, when executed on the processor cause the processor to perform operations comprising:
receiving user input requesting AI analysis of examination parameters;
initiating the AI analysis of the examination parameters in response to the receiving user input requesting AI analysis of examination parameters to generate an AI report;
storing the AI report in memory;
linking the AI report with an examination; and
in response to receiving a delete request before the examination has been saved, unlinking the AI report from the examination while maintaining the AI report in memory.

15. The apparatus of claim 14, the operations further comprising:
in response to receiving a terminate request before an AI report has been received, terminating the AI analysis; and
in response to receiving a terminate request after an AI report has been received, prohibiting terminating the AI analysis.

16. The apparatus of claim 14, the operations further comprising:
prohibiting unlinking of the AI report from the examination record after the examination has been saved.

17. The apparatus of claim 16, wherein the prohibiting unlinking comprises deactivating a delete AI report icon.

18. A computer readable medium storing computer program instructions, which, when executed on a processor, cause the processor to perform operations comprising:
receiving user input requesting AI analysis of examination parameters;
initiating the AI analysis of the examination parameters in response to the receiving user input requesting AI analysis of examination parameters to generate an AI report;
storing the AI report in storage;
linking the AI report with an examination record wherein a link is created to manipulate the AI report; and
in response to receiving a delete request of the AI report before the examination record has been saved, unlinking the AI report from the examination record wherein deleting the link while maintaining the AI report in storage.

19. The computer readable medium of claim 18, the operations further comprising:
in response to receiving a terminate request before an AI report has been received, terminating the AI analysis; and
in response to receiving a terminate request after an AI report has been received, prohibiting terminating the AI analysis.

20. The computer readable medium of claim 18, the operations further comprising:
prohibiting unlinking of the AI report from the examination record after the examination has been saved.
